(19) United States
(12) Patent Application Publication
(10) Pub. No.: US 2016/0296158 A1
(43) Pub. Date: Oct. 13, 2016

(54) NON-INVASIVE MULTI-FREQUENCY OXYGENATION SPECTROSCOPY DEVICE USING NIR DIFFUSE PHOTON DENSITY WAVES FOR MEASUREMENT AND PRESSURE GAUGES FOR PREDICTION OF PRESSURE ULCERS

(71) Applicant: DREXEL UNIVERSITY, Philadelphia, PA (US)

(72) Inventors: Elisabeth Papazoglou, Yardley, PA (US); Leonid Zubkov, Feasterville, PA (US); Michael S. Weingarten, Penn Valley, PA (US); Michael T. Neidrauer, Drexel Hill, PA (US); David Diaz, Perth Amboy, NJ (US)

(73) Assignee: Drexel University, Philadelphia, PA (US)

(21) Appl. No.: 15/037,778

(22) PCT Filed: Nov. 21, 2014
(86) PCT No.: PCT/US2014/066946

§ 371 Date: May 19, 2016

Related U.S. Application Data
(60) Provisional application No. 61/906,894, filed on Nov. 21, 2013.

(51) Int. Cl. A61B 5/00; A61B 5/1455

(52) U.S. Cl. CPC A61B 5/445; A61B 5/0075; A61B 5/14551

(57) ABSTRACT

Multi-frequency Diffuse Photon Density Wave (DPDW) methodology at near infrared wavelengths is used to non-invasively measure the optical absorption and reduced scattering coefficients of tissue at depths of several millimeters to quantify the depth and degree of tissue damage. A digitally-controlled stepper motor and variable RF generator enable the user to select the distance between the light source and detector and the modulation frequency through software. This allows for the collection of virtually unlimited number of data points, enabling precise selection of the volume and depth of tissue that will be characterized.

### CROSS-REFERENCE TO RELATED APPLICATIONS

The present application is a 371 application of International Application No. PCT/US2014/066946, filed Nov. 24, 2014. Which claims the benefit of U.S. Provisional Application No. 61/906,894 filed Nov. 21, 2013, the disclosures of which are incorporated herein by reference in their entirety.

### FIELD OF THE INVENTION

The present invention relates to a frequency domain near infrared absorption device for assessing deep tissue optical properties enabling, for example, the assessment of oxygenation of wound tissue, indicative of wound healing.

### BACKGROUND OF THE INVENTION

A variety of instruments based on the diffuse propagation of Near Infrared (NIR) photons due to multiply scattered light have been used to obtain clinically meaningful information about living tissue, such as tissue oxygenation. Such devices rely on optical fibers to transport the incident and scattered lights; however, the fiber optical probe is in contact with the tissue under examination. One of the key advantages of these laser technologies is their non-invasive nature. However, this advantage is negated by the fact that the fiber optical probe contacts the tissue under examination.

“Optical Properties of Wounds: Diabetic Versus Healthy Tissue,” IEEE Transactions on Biomedical Engineering, 53(6), 1047-1055 (2006), describes a frequency domain NIR instrument with one source position, four detector channels, three wavelength diode lasers (λ=685 nm, 785 nm and 830 nm) and a source modulation frequency of 70 MHz for assessing the early healing process of wounds in healthy and diabetic animals. The device enables the detection of oxygenated, deoxygenated hemoglobin and amount of blood. The wound healing assessment is further enhanced with the added capability to vary sensor penetration depth by adjusting the probe design. As illustrated in FIG. 1, the device included a 70 MHz RF signal with stable phase provided by RF generator block 1. The 70 MHz signal supplied the IQ demodulators of the four detectors 6-9 and modulated the emission light of three laser diodes 2. Two 1×2 optical switches and their drivers for optical switch 3 are were controlled using software executed on the illustrated personal computer 11 with control digital signals 12 being provided via data acquisition board (DAQ) 10. Light from the optical switch 3 was fed to 62.5 micron source fiber inserted in a probe 4 that was used to illuminate the experimental animal 5, one wavelength at a time. The scattering light was picked up by the four IQ detector bundle fibers 6-9. The detected electrical RF signal was then amplified by a first amplifier, filtered by a bandpass filter at 70 MHz, further amplified by a second amplifier, and fed to an UQ demodulator (not shown). The outputs of the IQ detectors 6-9 were the cosine (I) and sine (Q) low frequency components of amplitude and phase shift relative to the reference signal from the RF generator 1. These signals were further digitized by a 16-bit DAQ 10 for processing by the computer 11.

However, the device of FIG. 1 collects the light scattered from the tissue site using optical fibers that are in contact with the tissue surface and transfers this light to optical detectors 6-9. Such contact with the wound is undesirable and the contact probe provides no flexibility in rearranging the detector fibers and optimizing the setup for wounds of different surface areas, shapes, and depths because one source and four detector fibers are permanently inserted in a rectangular piece made of plastic, Teflon, or silicon.

Leonardi et al. describe in US 2006/0155193 another method for using a near infrared spectroscopy device to assess burn injuries. Leonardi et al. purport to use broadband white light and measure the intensity of the reflected light using a CCD. However, this device cannot obtain absolute values of absorption scattering coefficients but instead obtains relative changes. Moreover, the probe also must penetrate into the burned skin, which is generally undesirable.

There are many medical applications where it would be preferable or necessary not to touch the injury or wound site. Since the principle of operation of an fNIR device is to register the light scattered from the tissue, contact is not a limiting factor for the success so long as the light may be captured and its origin in the tissue accurately tracked.

Those skilled in the art will appreciate that in the field of NIR devices, the non-contact Continuous Wave (CW) method has been used for rapid and accurate acquisition of large data sets of tissue optical properties to reconstruct 2D or 3D images, for example, in breast imaging to detect tumors. Depending on the desired tissue volume to be covered (usually around 1 liter) and the required resolution (typically 0.2-1.0 cm), a very large number of measurements (from 10³ to 10⁵) are needed. A CCD camera is the most common device for reconstruction and is coupled with high quality lenses to achieve coverage of a substantial volume of tissue instead of an experimental probe with fibers, thus allowing for continuous wave (CW) measurements.

A frequency domain NIR device with a remote probe has been implemented for image reconstruction work and is based on a CCD camera coupled to a gain-modulated image intensifier with Fast Fourier Transform. This device is described by Godavarty et al. Physics in Medicine and Biology, 48,1701-1720 (2003), and by Gurfinkel et al. J. of Biomedical Optics, 9, 1336-1346 (2004). However, this device is very expensive and must be used on an optical table with very stable temperature and humidity conditions. As such, it is not suitable for clinical use.

A non-contact device having the same sensitivity and improved robustness compared to devices that must contact the wound is desired for many reasons. For example, a non-contact device may be used to obtain data from practically any wound and burn and, by not touching the injured skin, the measurements do not cause any pain or contamination of the wound. Other benefits of a non-contact device include the ability to maintain a sterile environment within measurements without a need for intermediate steps for sterilization, the elimination of operator variability due to differing contact pressures, and the ability to obtain measurements faster by a single operator. A non-contact device also may be mounted on a hyperbaric oxygen chamber for monitoring the status of a wound during and after treatments.

Several different types of frequency domain near infrared (fNIR) devices are known in the art. Currently three major experimental methods are used in the NIR range to measure absorption and scattering coefficients μ_{α} and μ′ₛ in multiply scattering tissues. The key difference among the various techniques lies in the source of incident light. The simplest and easiest method uses constant power lasers as, for example, in continuous wave (CW) devices. Since the power source for this method is constant, it is only possible to measure one parameter, the intensity of scattered light. Changes in this light intensity are measured as a function of source—detector separation ρ. In the case of CW devices, however, difficulties emerge when trying to separate absorption attenuation from scattering effects. CW methods give a composite “picture” of light intensity changes and cannot distinguish scattering from absorption. It is however possible to distinguish scattering from absorption phenomena using the following equation:

$$Φ ( r , t ) = \frac{{vM}_{0}}{4 π Dr} exp ( i kr ) exp ( - i ω t ) . \qquad ( 1 )$$

Equation (1) represents a solution of the diffusion equation for infinite homogeneous highly scattering media where Φ(r,t) is the photon fluence, v is the speed of light in turbid medium, D=v/3μ′ₛ is the photon diffusion coefficient, μ′ₛ=μₛ(1−D) is the reduced scattering coefficient, g=(cos θ) is the mean cosine of the photon scattering angle, and μₛ is the reciprocal of the scattering length. The complex diffuse wave wavenumber is a very important parameter=kᵣ+kᵢ. The square of the wave number k^{z}=(−3μ_{α}μ′ₛ+iωtμ′ₛ) is an expression where both coefficients μ_{α} and μ′_{α} are represented in an almost symmetrical expression and contribute in a similar way on the measured photon fluence Φ(r,t).

Both time resolution spectroscopy and the frequency domain technique are able to determine μ_{α} and μ′ₛ simultaneously. In the case of time resolved spectroscopy, a sequence of very short light pulses falls on the tissue under investigation and the broadening and shape of light impulses scattered from the tissue is analyzed. Although a wealth of information can be obtained, this method is complex and expensive and is difficult to implement in a routine clinical setting.

Time resolution spectroscopy (TRS) instruments also are able to obtain high quality information about the optical properties of the tissue from the broadening of very short light pulses after their propagation in tissue.

Although rich in information obtained, this method is complex and expensive and difficult to implement in a routine clinical setting.

The frequency domain technique with single modulation radio frequency RF of the incident light and variable source—detector separations can be used to simultaneously assess μₛ′ and μ_{α} of tissue, with a simpler and more cost effective device. Frequency domain devices measure directly two parameters: a) the intensity I(r,t) of scattered light, as in the case of CW methods, and b) the value of the phase shift Δφ, a parameter not obtained in CW methods.

The phase shift is a result of the light modulation in that there is a shift between the RF of modulated scattered light compared to the phase of the RF oscillator which is used for modulation. The phase shift Δφ occurs because of the diffusive aspects of light propagation in tissue representing multiple light scattering phenomena. For typical optical measurements where light enters the tissue through the skin and leaves the tissue at distance ρ from the entry point, the real path of light R in the tissue is R˜(10−20)*ρ by reason of the diffusion propagation of light. Fitting the experimental values of intensity and phase shift to the solution of the diffusion equation allows simultaneous determination of μₛ′ and μ_{α} in tissues. For these reasons, it is believed that the frequency domain technique will provide the most suitable non-contact device and have disclosed such a device in the above-referenced related patent application.

It is therefore an object of the present invention to validate a near infrared spectroscopy (NIRS) technology for detecting deep tissue injury (DTI) and distinguishing it from superficial stage I pressure ulceration using non-invasive optical technology to objectively measure the health of subsurface skin layers, subcutaneous fat and muscle so as to enable the early detection of DTI pressure ulcers before they are clinically visible. It is specifically desired to develop a simple, inexpensive, non-invasive multi-frequency NIRS system capable of quantifying the depth and extent of underlying tissue damage in skin and subcutaneous tissue at depths ranging from 500 microns to 10 mm before it is clinically apparent.

### SUMMARY OF THE INVENTION

A device for measuring tissue damage includes an opto-electronics module including an RF signal source that modulates the intensity of at least one laser diode to provide modulated light at different frequencies, and an optical switch that sequentially switches between respective laser diodes and a no light position. An optical probe applies the modulated light from the opto-electronics module to the tissue of a subject. In use, the optical probe applies the modulated light at a constant modulation frequency at multiple source—detector separations or applies modulated light at multiple modulation frequencies at a single source—detector distance so as to provide increased sensitivity at different depths in the tissue. At least one detector detects light scattered by the tissue in response to the modulated light, and a processing unit responsive to outputs of the at least one detector calculates optical properties and hemoglobin concentrations within the tissue for correlation with a state of the tissue at different depths beneath the skin. The probe may be a non-contact probe with source lenses and detector lenses for applying the modulated light to the tissue in a non-contact manner and detecting scattered light in a non-contact manner, respectively. The probe may also be a contact probe that has first optical fibers that apply the modulated light directly to the tissue of the subject and second optical fibers that contact the tissue of the subject to detect the scattered light. The probe also may be a semi-contact probe having source lenses for applying the modulated light to the tissue in a non-contact manner and optical fibers that contact the tissue of the subject to detect the scattered light.

The device may be used to detect pressure ulcers and other conditions characterized by decreased vascular flow. In an exemplary embodiment, the optical probe is inserted into a pad and prisms are affixed to an end of each of the optical fibers to turn light from the optical fibers 90 degrees through a hole in a side of the pad arranged to contact the subject.

Applications of such a device include assessment of wound healing, pressure sores, ischemia for various diseases and their complications. The device also may be used for chronic wound healing and burn treatment, to evaluate the efficiency of hyperbaric oxygen treatments, or to evaluate the effectiveness of wound and burn gels, scaffolds, and other treatment modalities.

### BRIEF DESCRIPTION OF THE DRAWINGS

FIG. 1 illustrates a prior art frequency domain NIR device that uses a contact probe to measure the scattering coefficient μₛ′ and absorption μ_{α}.

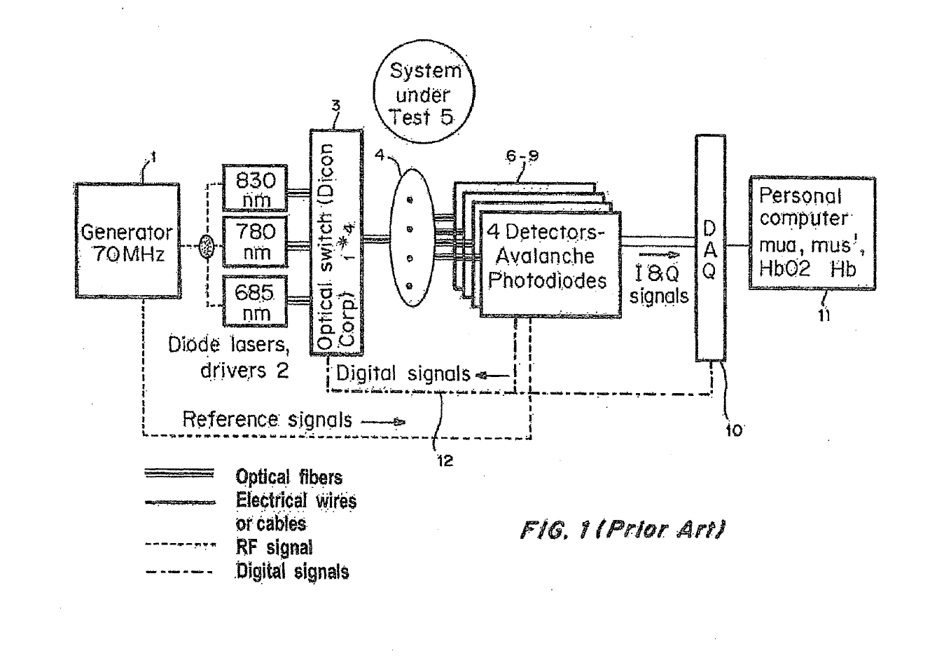

FIG. 2a illustrates a high level block diagram of a non-contact spectroscopy device in accordance with the invention.

FIG. 2b illustrates a high level block diagram of a contact spectroscopy device in accordance with the invention.

FIG. 2c illustrates a high level block diagram of a semi-contact spectroscopy device in accordance with the invention.

FIGS. 3a and 3b are graphs illustrating the set up for the non-contact (a) and semi-contact (b) configurations for the using the spectroscopy device.

FIG. 4 illustrates a contact configuration set up using eight source fibers and a singled detector fiber embedded in a Teflon probe.

FIG. 5 illustrates a detailed schematic of a frequency-domain near infrared system for a contact configuration where 2 APD detectors with their corresponding IQ demodulators are used to increase the number of source—detector separations.

FIG. 6 illustrates an overview of components in a multi-frequency NIRS system in a contact configuration and a flow of operation in accordance with the invention where the dashed lines indicate digital signals from the DAQ card and laptop and the solid lines represent analog signals.

FIG. 7 illustrates an overview of components in a multi-frequency NIRS system in a non-contact configuration and a flow of operation in accordance with the invention where the dashed lines indicate digital signals from the DAQ card and laptop and the solid lines represent analog signals.

FIG. 8 illustrates an overview of components in a multi-frequency NIRS system in a semi-contact configuration and a flow of operation in accordance with the invention where the dashed lines indicate digital signals from the DAQ card and laptop and the solid lines represent analog signals.

FIG. 9 is a graph of the linear relationship between the logarithm of the detected amplitude versus the logarithm of the incident optical power as measure by the spectroscopy device.

FIG. 10 is a graph illustrating offset stability test results for the spectroscopy device where all offsets were less than 1.4 mV.

FIGS. 11a and 11 b are graphs illustrating the results of a one hour stability test using a solid phantom where the standard deviation for amplitude is 0.9% and the phase shift standard deviation is 0.003 radians (0.2 degrees).

FIGS. 12a and 12b are graphs illustrating the stability of absorption and reduced scattering coefficients from day to day for a period of 6 days (<3% deviation) using the spectroscopy device.

FIGS. 13a and 13b illustrate absorption and reduced scattering coefficients obtained for multiple modulation frequencies using a multi-distance approach.

FIGS. 14a and 14b are graphs illustrating μs′ increasing as INTRALIPID concentration increases while μa stays relatively unchanged for measurements taken at 100 MHz using 685 nm laser light.

FIGS. 15a and 15b are graphs illustrating an increase in pa as ink concentration increases while μs′ stays relatively unchanged at each frequency.

FIGS. 16a and 16b are graphs illustrating scattering results from a 2 factor ANOVA analysis where μs′ increased after a deep burn but did not change after a superficial burn.

FIGS. 17a and 17b are graphs illustrating absorption results from a 2 factor ANOVA analysis where μa decreased immediately after a deep burn and gradually after a superficial burn.

FIGS. 18a is a cross-sectional view and 18b is a top view of the probe where optical fibers are glued to right-angle prisms and enclosed by a protective metal cylinder, all embedded within a silicone pad. The dashed line represents the path of light through the probe.

FIGS. 19a-19d are illustrations of tissue cross-sections illustrating the classification of pressure ulcers.

FIGS. 20a and 20b are graphs showing [HbO] steadily decrease from baseline values after pressure is applied to the sacral area when the patient moves from lateral to supine position. Inversely, [Hb] goes up when blood blow is restricted and comes back down when pressure is released.

### DETAILED DESCRIPTION OF ILLUSTRATIVE EMBODIMENTS

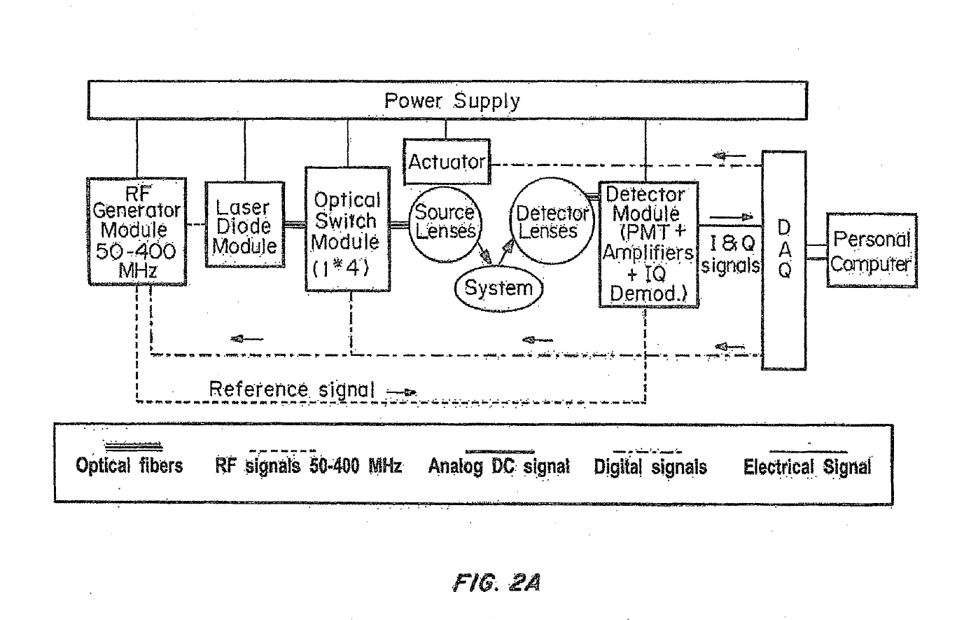

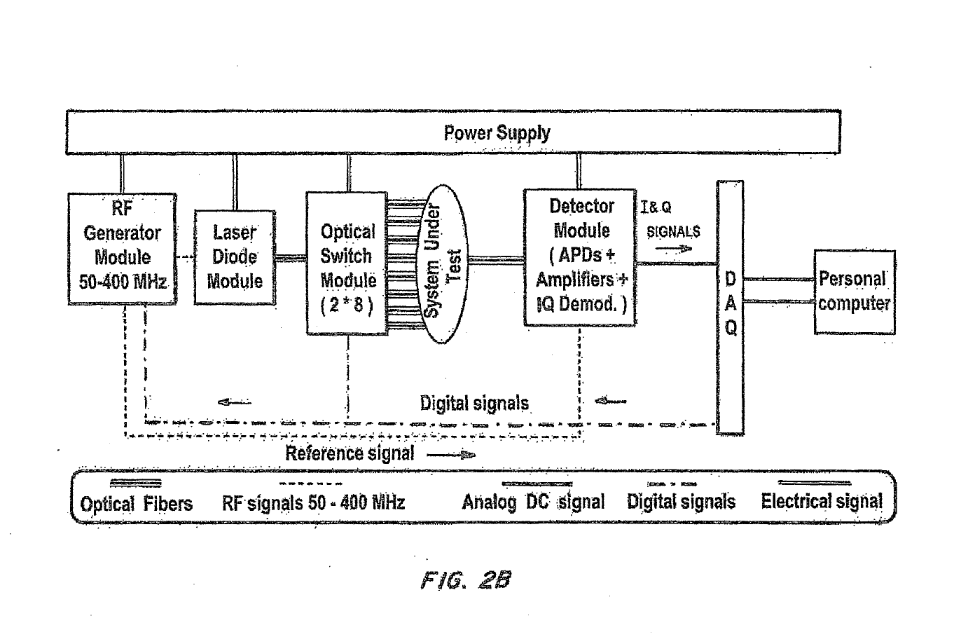

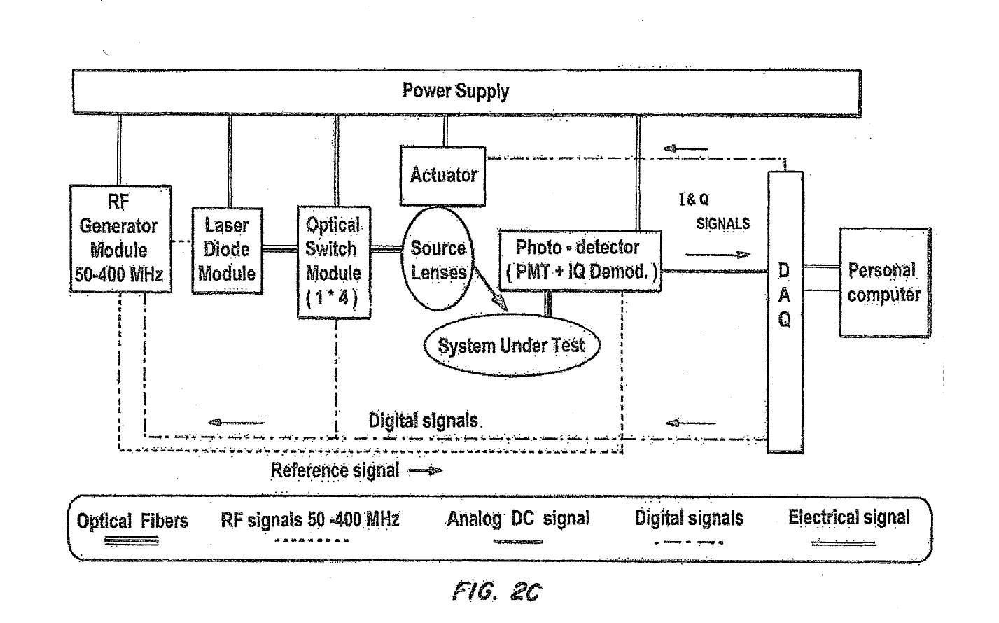

A detailed description of illustrative embodiments of the present invention will now be described with reference to FIGS. 2-21.

The method of Diffuse Near Infrared Spectroscopy (DNIS) allows the determination of the optical properties, specifically the absorption coefficient (μa) and reduced scattering coefficient (μs′) of non-homogeneous, strong light scattering media. Human and animal tissues can be analyzed quite accurately using the diffusion approximation of DNIS. Several non-invasive optical experimental techniques widely used for medical applications differ mainly by the type of incident light that illuminates the human or animal tissue. If frequency domain devices are considered where the incident light of NIR lasers is modulated by only one radio (RF) frequency, the absorption coefficient may be determined by measuring how the intensity of light scattering and the phase of registered light changes as a function of the distance between the light sources (mostly source fibers) and the detector fibers. The source and detector fibers are inserted in fixed positions on an experimental probe, usually made from a plastic semi-flexible material. A number of different combinations in source—detector distances is possible and gives rise to different device configurations, for example, 1 source—4 detectors, or 2 sources—2 detectors. In both of these cases, four experimental points are measured at four distinct source—detector separations. Typically, the probe is placed in full contact with the surface of tissue under investigation, which is illuminated through the source fibers. The required condition is that all fibers must be in contact with the tissue. The light registered by the detectors fibers consists of light that underwent multiple light scattering and as a result is propagating back to the surface; this corresponds to the semi-infinite geometry of the diffusion approximation.

The Photon Density Wave Methodology (PDWM) is widely implemented in different diagnostic medical applications for determining tissue optical properties. Usually, devices that are modulated by a single RF frequency light delivered to the tissue by one or several source optical fibers that are inserted in an experimental probe that is in contact with the tissue are used. The scattered light coming back from the tissue is collected by the detector fibers that are placed in the same probe as the source fibers, at various distances from them to enable illumination of tissue at different depths. The Diffusion approximation allows calculation of the optical properties of tissue from the experimentally measured amplitude and phase shift as a function of the distances between the source and detector fibers.

In the device described herein, however, the incident light from a laser-diode can be modulated at different RF frequencies. The multimode optical fiber delivers the laser emission to focused lens that are mounted at a moving part of a linear digital actuator. The source lens focuses incident light on the tissue surface. The relay lens is mounted at a distance an equal focal length from the tissue surface. In this condition, the relay lens transfers the image of small tissue surface on the detector fiber tip and, therefore, the scattered light from this small surface falls into detector fiber. The source—detector separations can be changed by applying the digital signal to the linear actuator that moves the source lens and consequently the source light spot on the tissue surface relative that observed by the relay lens tissue surface. The digital signal duration determines the step size in the source—detector separations. The amount of experimental data points that can be collected at various distances between the source and detector is now indefinite (very large practically). The source—detector separations can vary from very small (microns or smaller) to very large distances simply and inexpensively. The large amount of experimental data can help to improve the fitting of experimental data to the analytic solution of the diffusion approximation at relative large distances and particularly importantly allows one to obtain the reliable optical properties at the small distances where the diffusion approximation does not work.

The scattered light through a multimode fiber is sent to a detector block with a photomultiplier (PMT) tube or an avalanche photodiode (APD) as a detector element. The PMTs have a high photocathode sensitivity and can register high frequency optical signals without distortions. The set of amplifiers and IQ demodulators with wide frequency response used for amplification and creation of a low frequency IQ signal allow for calculation of the amplitude and phase shift of the scattered light.

A number of medical applications demand the accurate measurement of optical properties at small depths inside tissue that correspond to small source—detector separations, where the phase shift of scattered light is extremely small. The employment of different modulation frequencies allows a significant increase in the accuracy of the measured phase shift in particular at the higher frequencies. At the same time, the diffusion approximation allows the inventors to determine the optical properties from measurement at only one source—detector separation if the modulation frequency is changed. The one source—detector separation corresponds to measurements in small tissue volume with a small variation in tissue depth. That improvement is particularly important for the determination of burn depths.

The detector is thus invaluable for measuring burns, wounds, pressure ulcers, and skin lesions (carcinomas, scars etc.). The device is not sensitive to ambient lighting conditions and can assess from a few microns to several cm. Such distances are not currently possible with existing devices for example laser doppler systems.

### Principles of Operation

In turbid media, such as biological tissue, light propagation can macroscopically be described as a diffusive process. Diffuse near infrared spectroscopy (DNIRS) allows tissue to be non-invasively analyzed by measuring its optical absorption and reduced scattering coefficients (μ_{α} and μₛ′). In general, the absorption coefficient of tissue (μₐ) provides information about perfusion (hemoglobin content and oxygenation) while its reduced scattering coefficient (μₛ′) provides information about the structure and composition of the probed tissue. The optical absorption of hemoglobin decreases by an order of magnitude at red and near infrared (NIR) wavelengths (650-950 nm) when compared to other visible wavelengths. This behavior provides a “diagnostic window” that allows the determination of tissue optical properties at depths ranging from millimeters to centimeters.

The absorption spectra of oxy-hemoglobin, and deoxy-hemoglobin are distinct at NIR wavelengths, and therefore the absolute concentrations of each chromophore can be determined if the optical absorption coefficients are known at two or more NIR wavelengths. Oxyhemoglobin concentration [HbO₂] and deoxyhemoglobin concentration [Hb] can be calculated from the values of the optical absorption coefficient (μ_{α}) using the following equation:

ε_{Hb}^{λ}[Hb]+ε_{HBO2}^{λ}[HbO₂]+μ_{a ,H2O}^{λ}[% H₂O]=μ_{a,measured}^{λ} (Eq. 1)

where λ=685, 830 nm, ε_{Hb}^{λ} and ε_{HBO2}^{λ} are the molar extinction coefficients of deoxy- and oxyhemoglobin, μ_{a,H2O} the absorption coefficient of pure water, and [% H2O] is the percentage of water in the measured tissue, which is assumed to be 70%. Total hemoglobin concentration [Tot Hb] is calculated as the sum of [HbO₂] and [Hb]; oxygen saturation is calculated as [HbO₂]/[Tot Hb]×100.

The instrument described herein is able to detect changes in backscattered light intensity (I) and phase shift (Δφ) at two different wavelengths as a function of source detector separation (R) and/or modulation frequency (ω). Intensity modulated light propagates as diffuse photon density waves with a wave vector that is a function of both tissue optical properties and modulation frequency. This diffusion-based model of light propagation in tissue, can be used to calculate μₐ and μₛ′ by comparing the experimentally measured I and Δφ data to analytically derived model functions. The model functions had been previously derived from solutions of the photon diffusion equation which yield expressions for amplitude and phase as a function of R, ω, μₐ and μₛ′. Least-squares fitting methodology is used to fit the appropriate model functions to the collected amplitude and phase data. Phase or amplitude data alone provide sufficient information to extract absorption and reduce scattering coefficients. However, the use of both data simultaneously can significantly improve the robustness and fidelity of the fit.

Propagation of light in tissue can be described by the diffusion approximation only when the following assumptions are met:

1) μₛ′>>μₐ (i.e photons undergo multiple scattering events before they could be absorbed). This is the case in tissue where typical values of μa and μs′ are 0.1 and 10 cm⁻¹ respectively.

2) The source—detector separation distance (R) is greater than three times the transport length (l*). In other words, the minimum distance between source and detector fiber is greater than one to three transport lengths (ρ>3l*). The transport length or mean free path is the typical distance a photon must travel in turbid media before its scattering direction is randomized (l*=1/μₛ′). The reduced scattering coefficient, μs′=(1−g) μs, is defined, in terms of the scattering coefficient (μs) and the anisotropy of scattering, g, where g is defined as the average cosine of the scattering angle. After propagating more than one to three transport lengths most photons have undergone multiple light scattering (i.e. they are now at a different orientation from their incident direction) and may be described as diffuse. Calculation of optical properties at small depths is not possible using the commonly used diffusion-based model of DNIRS. Monte Carlo simulations will enable the extraction of tissue optical properties at depths ranging from 0.15 to 10 mm based on our multiple frequencies, single detector DNIRS system.

3) The light source/radiance is quasi-isotropic (i.e., the magnitude of light scattering/radiance is equal in all directions/angles). In the proposed system, light will be focused to a small point on the skin surface and will become effectively (quasi) isotropic after traveling one transport length.

4) The modulation frequency is much less than the frequency of photon collision (f<˜500 MHz). The proposed device uses frequencies ranging from 50 to 400 MHz.

Diffuse light propagation modeling allows measurements to be performed at multiple R using a single ω, as well as multiple ω at a constant R. Using multifrequency measurements at one source—detector separation allows for small tissue volume measurements with very small variation in tissue depth. This greatly improves the spatial resolution of DNIRS measurements, allowing one to more precisely select the volume and depth of tissue that will be characterized. An additional advantage of the new methodology is improved accuracy of phase measurements, because the phase delay Δφ is proportional to ω when R is constant. A higher ω will make phase changes easier to detect with higher accuracy.

To summarize, the system allows measurements of scattered light in two regimes:
Multi-distance measurements: Readings are taken at multiple source-detector separations using a constant modulation frequency. The depth of an NIRS measurement is proportional to the distance (R) between the light source and the detector. Thus, the obtained optical properties represent an average value across all R, and can only offer an overall value across a large volume of tissue.
Multi-frequency measurements: Readings are taken at a single distance using multiple modulation frequencies. The optical values obtained represent the average value across the same R. This greatly improves the spatial resolution of NIRS measurements, allowing one to more precisely select the volume and depth of tissue that will be characterized.

### Description of the Device

The device can consist of two separate parts: (1) a “box” containing the opto-electronic components such as lasers, photodetectors, and RF generators, and (2) an optical probe. The optical probe can have three different configurations: Non-contact, semi-contact, and fully contact. Each configuration has its own advantages and requires slightly different hardware from the rest. A non-contact system is ideal for assessing burns since it reduces the risk of infection and patient discomfort/pain, while a contact system provides faster measurements, easy set up, and can easily be used in a clinical setting. A description of the overall system will be given and differences in hardware and functionally between the different configurations will be described whenever relevant.

In a prototype embodiment, the “box” containing opto-electronics parts has been assembled within a standard 12″ rack with 6 shielded NIM box modules. The contents of each module are: 1. Power supply, 2. Radio Frequency (RF) Generator and amplifiers, 3. Laser-diodes (685 nm and 830 nm) with drivers, 4. Optical switch and Stepper motor driver, 5. Detector with amplifiers and IQ demodulator. A National Instruments USB-6251 M Series DAQ Card is used for data acquisition and digital control of several components. A personal computer equipped with LabVIEW and Matlab software provides the control interface for the overall system and analysis/fitting of experimental data to calculate the optical properties and hemoglobin concentrations within tissue. A high level block diagram of the different modules in the non-contact system is shown on FIG. 2a. FIGS. 2b and 2c illustrate a contact configuration and a semi-contact configuration, respectively.

### Power Supply Module

Different system components require different levels of voltage in order to operate. The power supply module converts the alternating current (AC) coming from the wall outlet into direct current (DC) that can be used to power the different components of the device. In an exemplary embodiment, this module provides voltages at 3.3, 5, 12, and 15 V.

### RF Generator Module

A locking programmable sine wave generator (LPO400A Novatech Instruments, Inc.) produces sinusoidal Radio Frequency (RF) signals ranging from 50 to 400 MHz by steps of 1 MHz. The generated signal is amplified and split using a two way RF splitter. One channel, called the local oscillator (LO), is kept as a reference. The second RF signal modulates the intensity of light emitted from the laser diodes. As the modulation frequency changes from 50 MHz to 400 MHz, the electrical components give a slightly different response per frequency, causing inconsistent modulation depths across different frequencies. To compensate for this difference, a variable gain amplifier (Analog Devices, AD8375) is used to maintain a constant modulation depth of laser radiation. The AD8375 variable amplifier can modify the amount of RF signal attenuation from 0 to 24 dB (with 0 dB representing the highest gain and 24 dB representing no gain). Both the RF generator and variable gain amplifier are controlled by digital signals generated by a data acquisition board (National Instruments, USB-6251) and laptop computer.

### Laser Diode Module

The RF signal coming out of the variable amplifier modulates the intensity of two semiconductor laser diodes of different wavelengths (685 nm and 830 mn). In order to obtain intensity modulated light, an AC signal must be mixed with the DC bias of the lasers using laser drivers. Small size laser drivers are not commercially available and were engineered in-house. The in-house laser drivers modulate the amount of current going into the laser diodes resulting in intensity modulated light. In addition, the drivers protect the laser diodes from excessive current damage. The design of the driver depends on the laser type; the drivers use an integrated circuit from Sharp or from ic-Haus and bias-tee from Mini-circuits.

### Optical Switch Module

### Non-Contact/Semi-Contact

For non-contact and semi-contact configurations, the optical switch module houses a 4×1 optical switch (DiCon Fiberoptics) which allows toggling between the two laser diodes and an offset (no light) position. The offset value is used to determine (and compensate for) the voltage read by the device when no incident light is being delivered. The optical switch cycles through the offset value (no light), then the 685 nm laser light, and finally the 830 nm lasers light for each individual distance/frequency. The switch is controlled by digital signals from the DAQ card and laptop computer. A variable optical attenuator (Oz Optics, Ltd) is connected in between each laser diode output and the corresponding optical switch inputs to provide control over the intensity of light delivered to the tissue. A 62.5/125 μm multimode optical fiber is used to deliver light from the optical switch to the source lens assembly which focuses the light into the tissue.

A digital actuator is used for changing the distance between the light source and detector. A stepper motor driver is used to digitally control the actuator. The duration of the digital signal determines the step size of the actuator. A step size of 0.25 mm is usually used to control the source—detector separation. Moving the source fiber significantly increases the number of source—detector separations and therefore the number of data points. The depth of each measurement (i.e: how far into the tissue is probed) is estimated to be approximately ⅓ to ½ of the distance between source and detector.

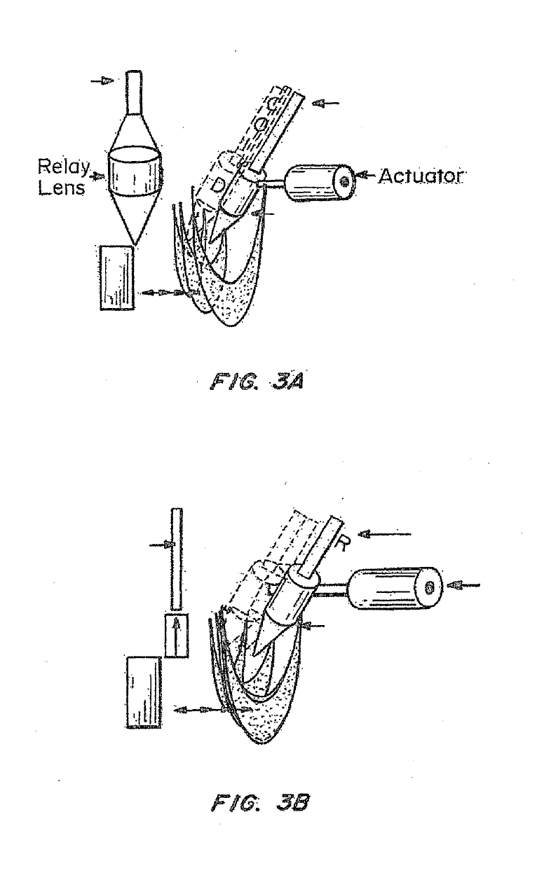

During non-contact measurements, a lens assembly first collimates and then focuses the laser light from the source fiber onto the skin at a location controlled by the digital actuator. A relay lens (Edmund Optics, M45-762) collects scattered light from a point on the skin surface and projects the skin image to a 62.5 μm diameter optical fiber which transports the detected light to a photodetector as illustrated in FIG. 3a. The semi-contact configuration has a similar set up than the non-contact except there is no relay lens. The backscattered light is collected by a detector fiber directly touching the optical system being probed as shown in FIG. 3b.

### Contact

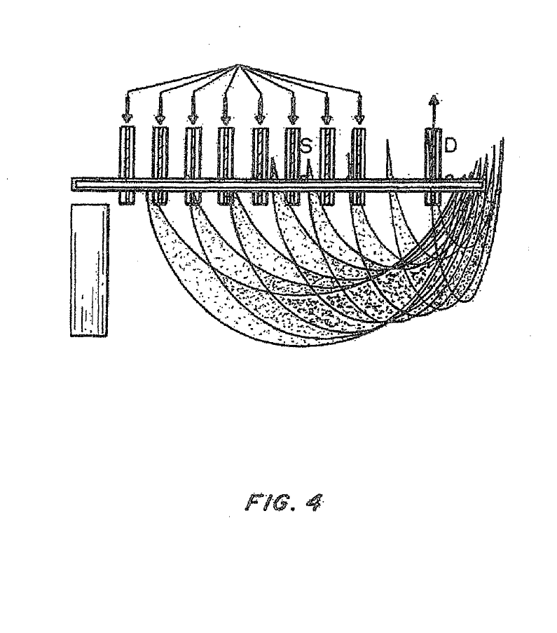

During contact measurements, the source and detector fibers are embedded into a rigid Teflon probe that comes into direct contact with the skin or optical system being probed. The incident laser light is delivered to the system by eight 62.5/125 μm multimode optical source fiber. Two optical fiber bundles with 1.0 mm diameters are used to deliver backscattered light from tissue to two avalanche photodiode (APD) modules (Hamamatsu C5658). In an exemplary embodiment, the contact configuration provides a total of 16 source—detector separations ranging from 4 mm to 19 mm. A 2×8 optical switch (DiCon Fiberoptics) enables the delivery of modulated laser light to each one of the source fibers by cycling through each laser and source fiber combination. FIG. 4 illustrates a contact set-up using a single detector fiber. Only one detector fiber is shown in FIG. 4 for ease of understanding.

An Avalanche Photodiode (APD) module (Hamamatsu, model 05658) is used because it has a wide range of operating frequencies (up to 1000 MHz) which enables the inventors to take advantage of the RF generator's full range of frequencies. The broad linear range of an APD combined with its ability to detect high levels of incident light without being damaged makes it an ideal detection system for this application.

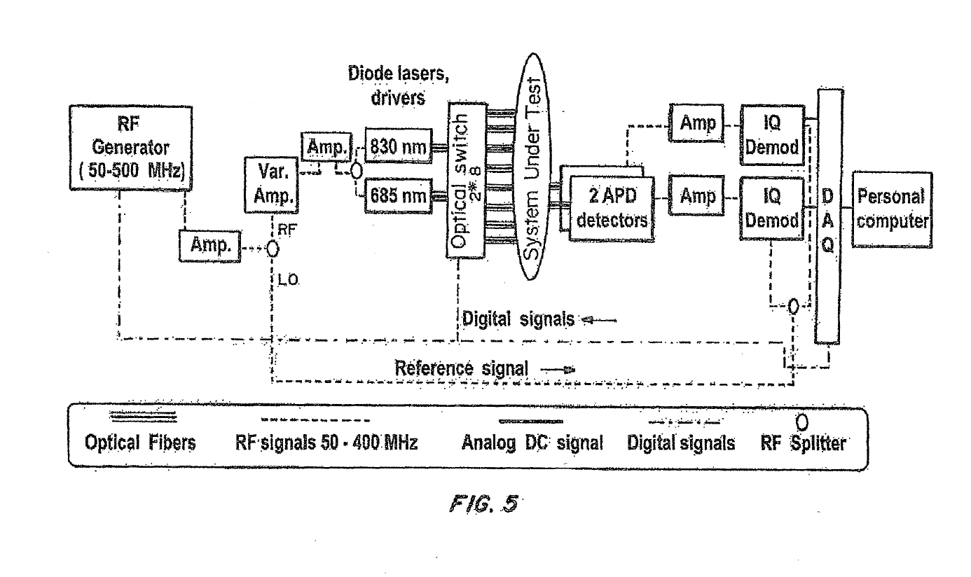

The electrical signal from the photodetector(s) is passed through two fixed amplifiers (Mini-Circuits, ZFL-500LN and Mini-Circuits, ZFL-500HLN). Then the signal is fed to an In-Phase/Quadrature (I/Q) demodulator (MERRIMAC IQM-9B-500), which compares the detected signal to the reference LO signal from the RF generator. The outputs of the I/Q demodulators are the cosine (I) and sine (Q) low frequency components of amplitude and phase shift relative to the reference signal. The I and Q signals are then passed through a low-pass filter (DC to 1.9 MHz), digitized by the data acquisition board (National Instruments), and recorded on a laptop computer. A detailed block diagram of the system in a contact configuration is illustrated in FIG. 5.

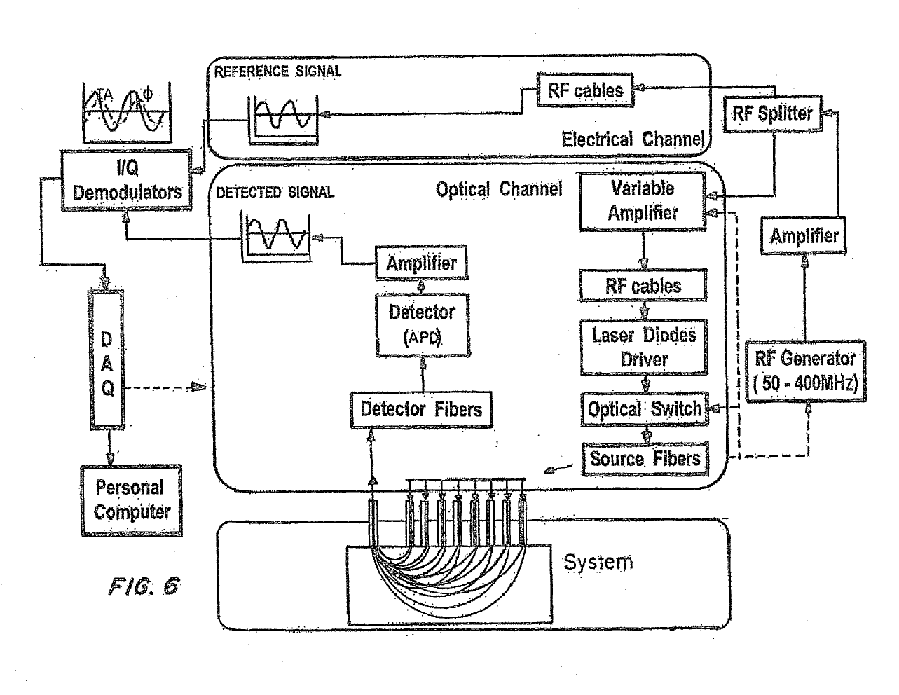

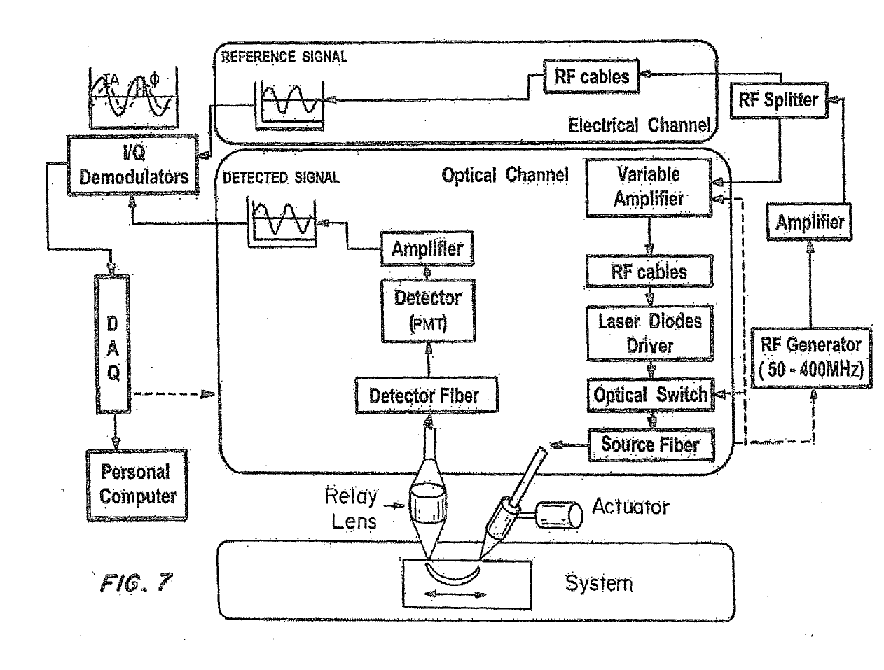

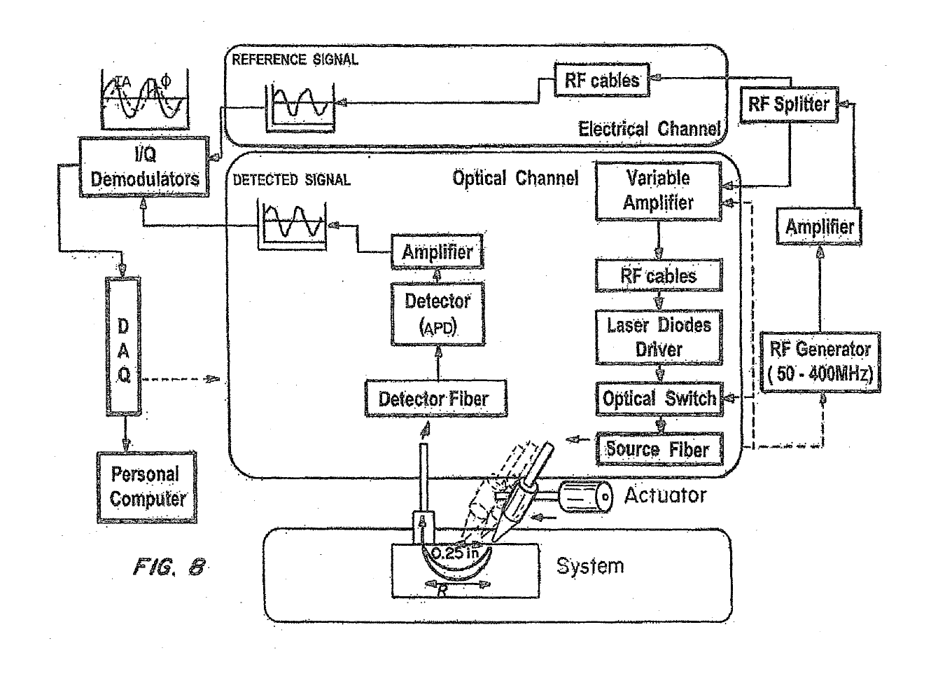

FIG. 6 provides a visual overview of all of the instrument components for the contact configuration and the flow of operation from module to module. For ease of understanding only one detector is shown. Similarly, FIG. 7 provides a visual overview of all of the main instrument components and flow of operation for a non-contact configuration, while FIG. 8 provides a visual overview of all of the main instrument components and flow of operation for a semi-contact configuration.

Those skilled in the art will appreciate that, when detecting pressure ulcers, a probe that requires contact with the skin has several advantages over a non-contact probe:

1. A contact probe allows measurement of skin (and subcutaneous tissue) under pressure. This is critical for pressure ulcer diagnosis because there is evidence that the response of tissue to an ischemia-reperfusion cycle is affected by pressure damage.

2. The contact probe will be easier to set up and use in a patient exam room. Results obtained from a non-contact probe are more likely to be affected by patient motion than a contact probe.

3. A contact probe may help with commercialization, because the manufacturer can sell a disposable sterile covering that must be replaced for each patient.

4. The contact probe will enable faster measurements because the optical switch can cycle through multiple fixed locations significantly faster than a digital stepper motor would.

### EXAMPLE 1

### Experimental Data and Device Calibration

### Detector Linearity Testing

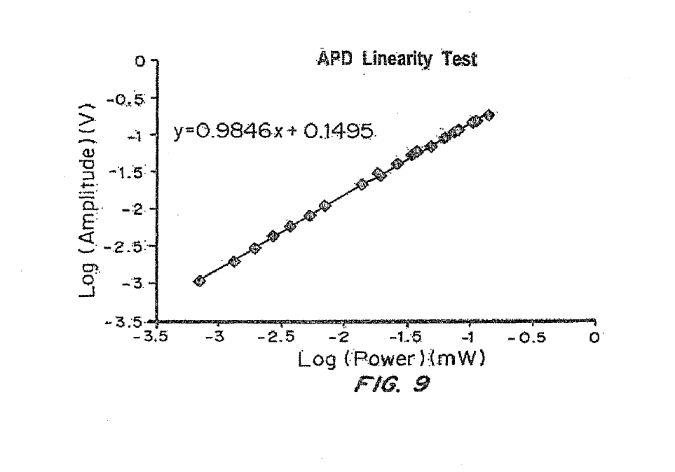

As with any optical detector, the APD module has a limited range where the electrical output signal is linearly proportional to the optical power of the incident light. Prior to in vitro or in vivo experiments, a calibration procedure is conducted to define the range of linearity. Any subsequent measurements which falls outside of the range of linearity is discarded and retaken after adjusting the amplitude of the signal using the optical attenuators. The results from the linearity test performed at a modulation frequency of 80 MHz are presented on FIG. 9. The linear range (slope of 0.98) observed for the APD indicates excellent detector linearity over the range of 1 to 185 mV.

### Offset Testing

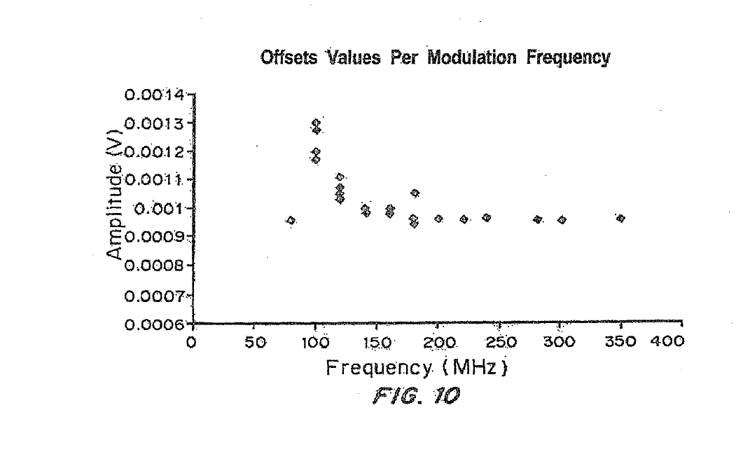

Offset values (i.e., voltage registered by the detector when there is no incident light) of the device were measured while changing the modulation frequency from 50 to 350 MHz. Results are shown in FIG. 10. The offset values were acceptable for all frequencies. Offsets are stable from day to day measurements.

### Stability

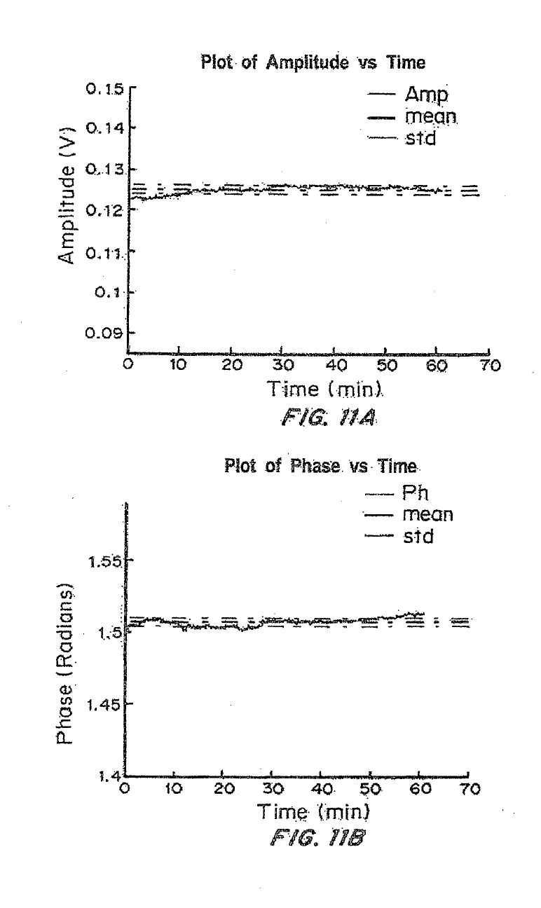

The ability for the instrument to provide consistent measurements throughout long measurement session that could last for more than 1 hour was validated using solid silicon phantoms. Measurements were obtained continuously at a single source—detector separation over a period of 1 hour using a modulation frequency of 70 MHz. The silicone optical phantoms used contained dispersed particles of titanium dioxide that act as scatters and carbon black to act as absorbers in order to mimic the properties of human tissue. Plots of amplitude and phase are shown in FIGS. 11a and 11b.

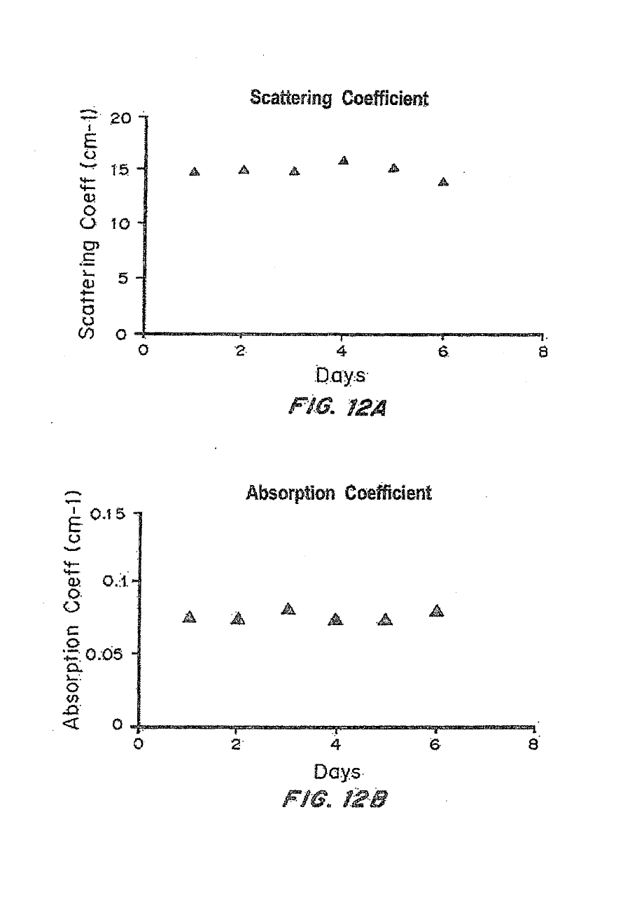

In order to assure consistent and stable results were obtained from day to day, routine measurements were taken on a solid silicon phantom using a multi-distance approach at a modulation frequency of 100 MHz. The day by day stability test is shown in FIGS. 12a and 12b.

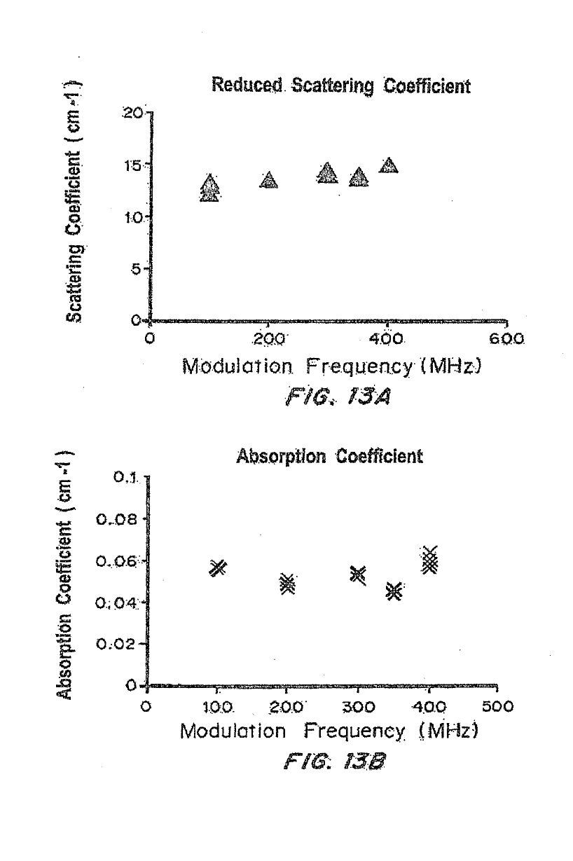

The optical properties of solid silicon phantoms were measured using multiple source-detector separations at different modulation frequencies. Optical properties close to the expected values (obtained values μ_{α′}=0.056 and μ′ₛ=13, expected values μ_{α′}=0.06 and μ′ₛ=14) were repeatedly obtained, as shown in FIGS. 13a and 13b. The standard deviation across the different frequencies for the optical properties is less than 9% with a standard error smaller than 3%.

### Titration Experiment

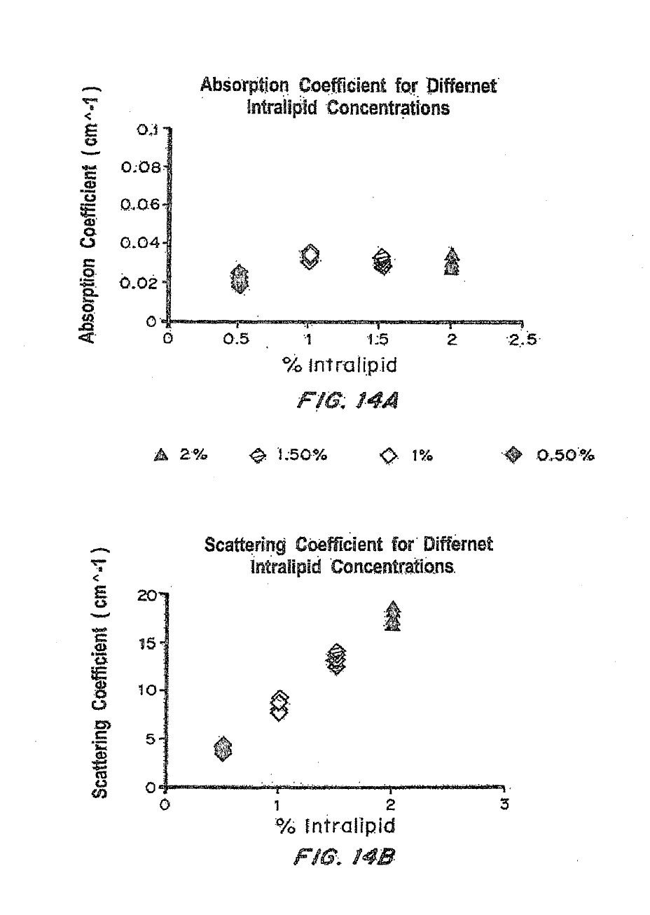

INTRALIPID, an emulsion of lipid droplets in water, is commonly used as an optical phantom because its light scattering properties are similar to living tissue. The INTRALIPID used in the experiments (LIPOSYN 20% from Abbott Laboratories) is diluted in deionized water to concentrations ranging from 0.5-2.0% to produce liquid phantoms with reduced scattering coefficients (μ′ₛ) similar to those in human tissue. A titration experiment was conducted where multiple dilutions of INTRALIPID solution were measured. As the concentration of INTRALIPID decreases, so does the number of scattering molecules, thus a decrease of the reduced scattering coefficient (μ′ₛ) is observed for lower INTRALIPID concentrations. Conversely, the number of absorber molecules is not affected by the dilutions and the absorption coefficient (μ_{α′a}) is expected to remain fairly constant. The results are shown in FIGS. 14a and 14b.

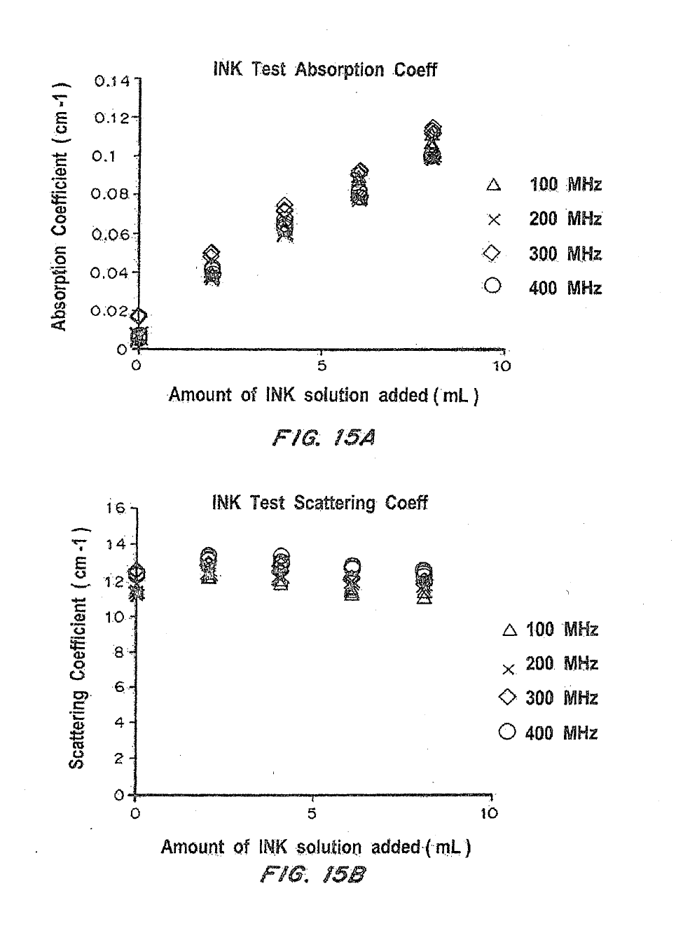

Moreover, Black India Ink (Higgins Corp) can be added to INTRALIPID solution to alter the optical absorption properties of the liquid phantom. The relative concentration of INTRALIPID to ink is approximately 10,000:1 to resemble properties of human tissue. An ink experiment was also performed were multiple dilutions of ink were added to 1000 mL of 1% INTRALIPID solution. As the concentration of ink solution increases, so does the number of absorbing molecules, thus an increase of the absorption coefficient (μ_{α′}) was observed. Conversely, the number of scattering molecules is not affected by the addition of the ink solution and the reduced scattering coefficient (μ′ₛ) was observed to remain fairly constant. The results are shown in FIGS. 15a and 15b.

### EXAMPLE 2

### Animal Study Results

An animal study for in vivo validation of the device was conducted. The purpose of the study was to differentiate superficial and deep burns using the optical data. Data analysis suggests a statistically significant difference between the optical properties of deep and superficial wounds. An in depth description of the results and study procedures follows:

### Study Procedures

A Yorkshire swine weighing approximately 25 kg was purchased and transported to the DUCOM facilities. The animal was acclimated for a period of one week. Baseline measurements of healthy intact animal skin were taken following the acclimation period. Anesthesia was induced by mask prior to measurements and the animal's hair was clipped to improve the accuracy and reliability of the measurements. The animal was intubated and mechanically ventilated to control the rate of breathing and reduce motion artifacts during measurements. After hair removal, eight wound sites were symmetrically marked in the dorsal area of the animal. Six days after baseline measurements, the same locations were used during the creation of the burns. A total of eight burns of two different depths (4 wounds per depth) were induced on the back of the animal (4 on each flank). The superficial and deep burn sites were staggered to correct for possible variations in optical properties due to anatomical differences from one location to the next. The wounds were inflicted by applying a cylindrical copper rod, 1-inch in diameter, with a temperature of 100° C. to the animal skin for a duration of 3 seconds (for superficial burns) and 20 seconds (for deep burns) with all pressure supplied by gravity. The metal rod was preheated to the desired temperature by immersing it in a bath of boiling water for one hour.

### Optical Measurements

The device used a semi-contact configuration in which the incident light was delivered via lenses and detected through an optical fiber in contact with the animal skin. Two different lasers (685 & 830 nm) were used to find the absorption (μₐ) and reduced scattering (μₛ′) coefficients of the probed tissue. Measurements were taken at two time points (immediately and 2 hours after burn) for each burn site using three different modulation frequencies (100, 150 & 200 MHz) at source-detector distances ranging from 4 mm to 10 mm.

### Results

The optical properties of deep and superficial wound sites where compared for baseline and post-burn measurements. The inventors obtained similar results in μₐ and μₛ′ for baseline measurements taken on different locations of the unburned animal skin (less than 5% difference). This indicated that all future wound locations would have tissue with similar μₐ and μₛ′ prior to inducing the burns.

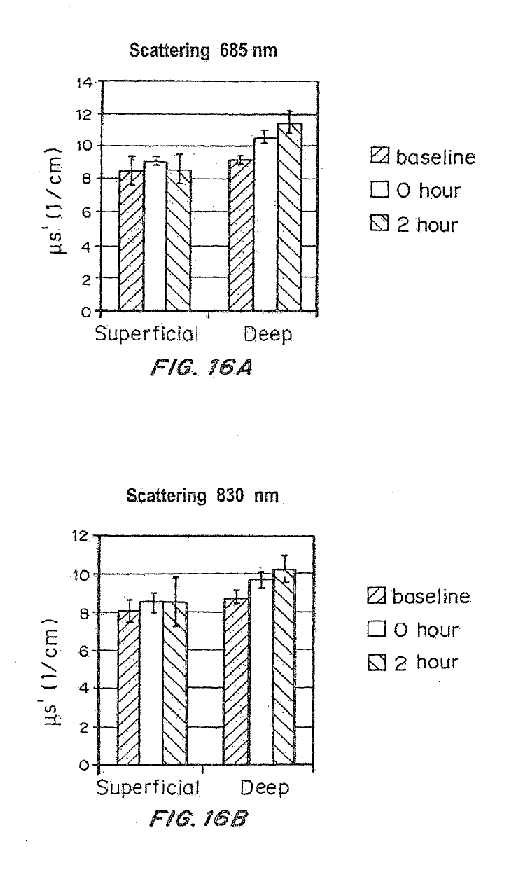

No significant difference was found across measurements of different frequencies. The average value across all frequencies for each site was used during data analysis. To identify a difference in optical properties between deep and superficial wounds, a two factor ANOVA with repeated measures was performed. There was a statistically significant difference between the μₛ′ of superficial and deep burns (p<0.05). Potentially, this could provide a simple way of wound differentiation. The μₛ′ provides information about the structure and composition of the probed tissue. In FIGS. 16a and 16b, it is shown that over time, μₛ′ stayed relatively unchanged for superficial burns but increased for deeper wounds. This trend is consistent with the relatively low level of structural change undergone by skin during a superficial burn and the large amount of tissue damage of deep burns.

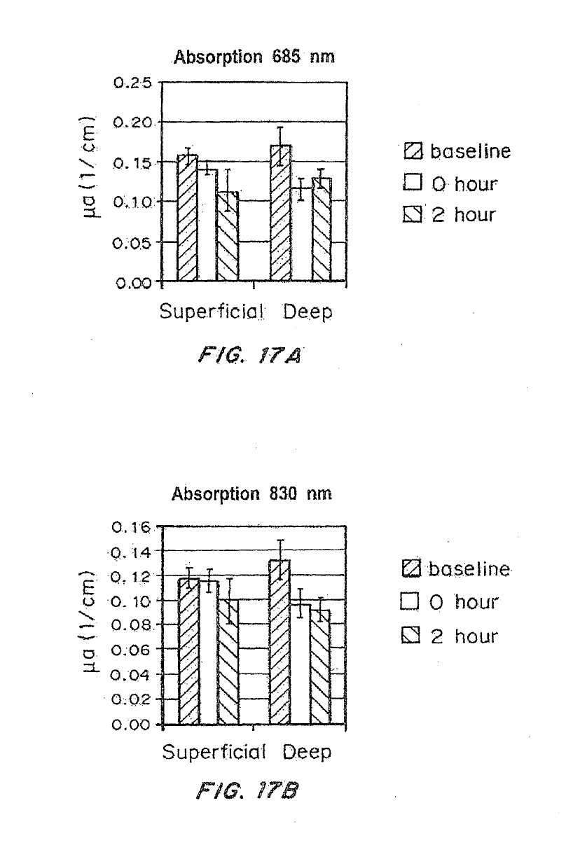

The μₐ provides information about hemoglobin content and oxygenation. The gradual decrease of μₐ in superficial burns is consistent with the expansion of burns for up to 72 hours described in the literature. This decrease shown in FIGS. 17a and 17b is due to a reduction in vessel density and reduced hemoglobin concentrations which in term reduce the amount of absorption that occurs in the probed tissue. The drastic decrease of μₐ observed in deep burns correlates with a larger change in blood vessel density and oxygenation of the tissue. Due to the large magnitude of the change in deep burns, the slow expansion of the burn is not very noticeable.

### Histology

Approximately four hours after the burn procedure, the animals were euthanized and full thickness excisions were taken from each burn area as well as adjacent healthy skin. Hematoxylin and cosin (H&E) staining was used to assess the depth of both superficial and deep burns. Burn depth was approximately 1 mm for all superficial wounds and 2 mm for deep burns.

### Applications of the Near InfraRed Technology

The use of near infrared (NIR) technology has been validated for detecting deep tissue injury (DTI) and distinguished from superficial stage I pressure ulceration using non-invasive optical technology to objectively measure the health of subsurface skin layers, subcutaneous fat and muscle. Such technology will be used for the early detection of DTI pressure ulcers before they are clinically apparent. There is considerable evidence that many pressure ulcers originate beneath the skin in subcutaneous soft tissue and/or muscle; however, the current clinical classification system for pressure ulcers and DTI relies on visual observation of the skin surface. The spectroscopy system described above provides an optical system that enables proper classification of existing pressure ulcers and DTI; such classification is critical because it governs treatment recommendations and hence the healthcare cost. Furthermore, the diagnostic technology may enhance the sensitivity and specificity of early pressure ulcer detection in comparison with currently used risk assessment tools.

Pressure ulcers (PUs) are a common complication for individuals with limited mobility, including the elderly, anyone recovering from surgery, and spinal cord injury patients. Pressure ulcers cause considerable harm to patients in acute care, long-term care, and rehabilitation facilities, hindering functional recovery while frequently causing pain and the development of serious infections. Pressure ulcers have been associated with an extended length of stay, pain, anemia, local infection, sepsis, gas gangrene, and mortality.

It is widely accepted that PUs are caused by excessive pressure and/or shear stress in soft tissue (skin, subcutaneous fat, and muscle) at vulnerable points near bony prominences, and there is considerable evidence linking blood vessel damage to pressure ulceration. The histological analysis of porcine and rodent animal tissue have indicated that reperfusion injury, which is cellular injury resulting from the reperfusion of blood into previously ischemic tissue, may cause microvessel damage and lead to ulceration of both cutaneous and subcutaneous tissues. Therefore, it is likely that impaired tissue oxygenation will serve as a useful indicator of the risk of pressure ulceration.

There are two hypotheses that describe how pressure ulcers propagate. In top-to-bottom hypothesis, ulcers start as damage to the microvessels of the upper dermis and spread to subcutaneous tissues if external pressure is not relieved. In contrast, the bottom-to-top hypothesis suggests that ulceration begins in subcutaneous skeletal muscle and grows upward to the skin, because the high metabolic activity of muscle makes it more sensitive to ischemia than skin It is likely that some ulcers form top-to-bottom and others form bottom-to-top; the optical method described herein will provide information about tissue health at both superficial and deep depths, enabling identification of either ulcer type.

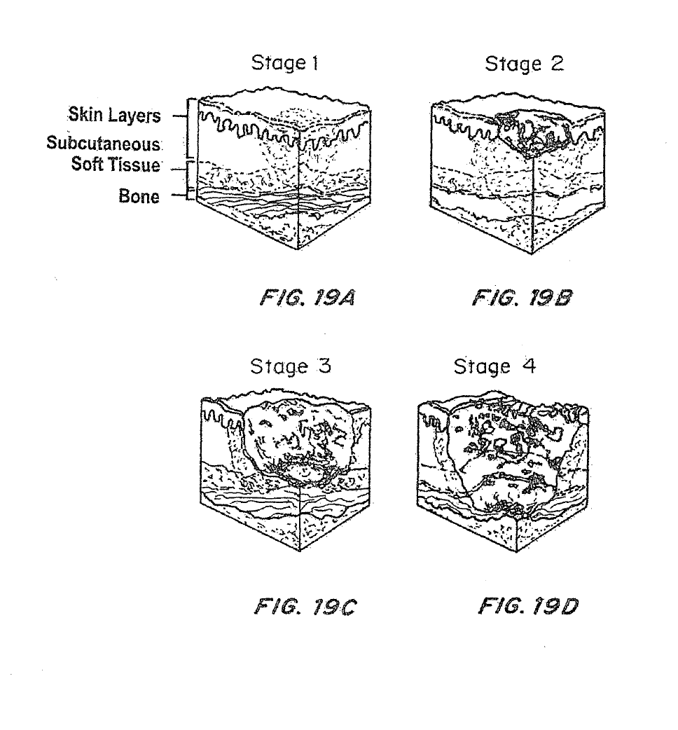

Proper classification of existing pressure ulcers is critical because it drives treatment recommendations. In the United States, the most widely used pressure ulcer classification system is the one developed by the National Pressure Ulcer Advisory Panel (NPUAP). It defines five categories for classifying PUs: stage I, II, III, and IV pressure ulcers, which refer to surface injuries of increasing depth and severity, and suspected DTI. A stage I pressure ulcer represents the least severe category, and is defined as “intact skin with non-blanchable redness” (i.e. redness that does not fade when touched). The fifth category, suspected DTI, represents potentially severe damage to subcutaneous tissue, and is defined as “Purple or maroon localized area of discolored intact skin or blood-filled blister.” As shown in FIGS. 19a-19d, Stage 2 has partial skin loss—epidermis, dermis or both; Stage 3 has full thickness skin loss but does not involve the fascia; and Stage 4 has full thickness skin loss with bone or muscle involvement. In practice, it is difficult to differentiate stage I PUs from suspected DTI, especially in patients with darker skin tones, because both manifest themselves as intact skin with redness or discoloration. Use of the noninvasive optical technology described above provides information about skin and subcutaneous tissue damage at multiple depths, up to 1 centimeter beneath the skin, and has potential to provide precise differentiation of stage I pressure ulcers from deep tissue injury.

The technology described herein may assist clinicians with early identification of pressure ulceration in all patients, before tissue damage is clinically apparent. This is especially critical with DTI, because significant sub-surface tissue damage may occur before redness or discoloration is apparent on the skin surface. When early tissue damage is detected, aggressive treatment protocols can be implemented to prevent further DTI and to reverse tissue damage by maximizing blood flow to the area. The capability of assessing a patient's pressure ulcer risk objectively, through measured data, will more precisely allow preventative measures to be instituted exclusively for the patients who need them.

Currently, clinicians classify pressure ulcers based on surface appearance and palpation. A quantitative, objective method of measuring the depth of tissue damage would enable precise differentiation of stage I pressure ulcers from DTI, allowing clinicians to give the appropriate treatment. However, there are no noninvasive methods of measuring microcirculatory hemoglobin oxygenation at depths of 1-4 mm. The multi-frequency DNIRS system described above is capable of measuring hemoglobin oxygenation at multiple depths up to 10 mm

Laser Doppler, Laser speckle contrast analysis (LASCA) and Hyperspectral imaging have been used in research studies as imaging modalities for the assessment of pressure ulcers. However, these methods cannot measure tissue at depths beyond 1 mm and therefore cannot be used to evaluate suspected deep tissue injury. Transcutaneous oximetry has been used to show differences in tissue oxygenation between patients with PUs and patients without PUs. However, it is difficult to use transcutaneous oximetry for routine measurements in clinical environments because the electrodes require 20-30 minutes of warm-up and stabilization time on a patient's skin before any reliable data can be obtained, and because the electrode site must be shaved and tape stripped to remove loose skin cells prior to data acquisition.

Diffuse Reflectance Spectroscopy, thermography, high-frequency ultrasound, optical coherence tomography, and magnetic resonance imaging have been used in research studies to assess pressure ulcer severity damage with mixed success; however, none of these technologies are commercially available as pressure ulcer assessment tools. These technologies have not gained clinical acceptance because of limitations in data quality or the practicality of using the device in a clinical environment.

A previously developed a frequency-domain DNIRS diagnostic system for evaluating the efficacy of diabetic foot ulcer treatments, is described in U.S. patent application Ser No. 13/125,116. However, this technology measures oxygenation in relatively large tissue volumes, i.e. it has poor spatial and depth resolution. Furthermore, it cannot be used to assess tissue damage at depths of less than 4 mm which prevents direct assessment of dermal health. By contrast, the non-contact, multi-frequency, optical system described above permits quantifying tissue damage at multiple depths.

### EXAMPLE 3

### Design of Probe for Non-Invasive Measurement

In exemplary embodiments, the probe described herein is used with a device the size of a small desktop computer, and the probe is gently placed on the surface of intact skin to assess tissue damage at multiple depths. Ideally, the probe could be manually held in place by a nurse at any anatomical location where DTI is suspected, and the measurement could be completed in less than one minute. The computer would immediately provide the clinician with a depth-profile of tissue oxygenation at depths ranging from 1-10 mm. The depth of bone within this range would be clearly indicated, and regions of impaired tissue oxygenation could be clearly seen.

In many cases, such a simple measurement protocol might not provide sufficient information to distinguish healthy from damage tissue with acceptable sensitivity and specificity. A more complex protocol may provide more information. When pressure is applied to the skin over bony prominences, tissue oxygenation is compromised. When the pressure is relieved, a transient increase of oxygenation (hyperemic response) is observed. Increased duration of the hyperemic response, caused by reperfusion injury to microcirculatory blood vessels, has been associated with patients at high risk of pressure ulcer development. However, these studies were all conducted using technologies that can measure only superficial tissue skin layers and could not be used to assess sub-surface DTI. Hyperemic response in the subcutaneous tissue of healthy subjects after rising from sitting position was observed using invasive subcutaneous oxygen electrodes, suggesting that a subcutaneous hyperemic response can be induced with external pressure. However, the electrodes are too invasive to be used with patients at risk of pressure ulceration.

For use in measuring deep tissue injury, the noninvasive probe described above touches the surface of the skin and measures tissue oxygenation at multiple depths. When optical fibers are used, the ends of the optical fibers in the probe are terminated with tight right-angle ferrules (from Fiberoptic Systems, Inc), and the ends of the fibers are embedded within a layer of silicone such that the tips are level with the rubber surface. When the region of interest is not bearing the weight of the patient, the probe will be manually applied to the skin by an operator or fastened in place with medical tape. When the region of interest is moved to a weight-bearing position, the silicone mat will prevent the fibers from harming the patient's skin and will protect the fibers from the weight of the patient. The mat will be covered with a sheet of transparent sterile Tegaderm™ that can be replaced between patients.

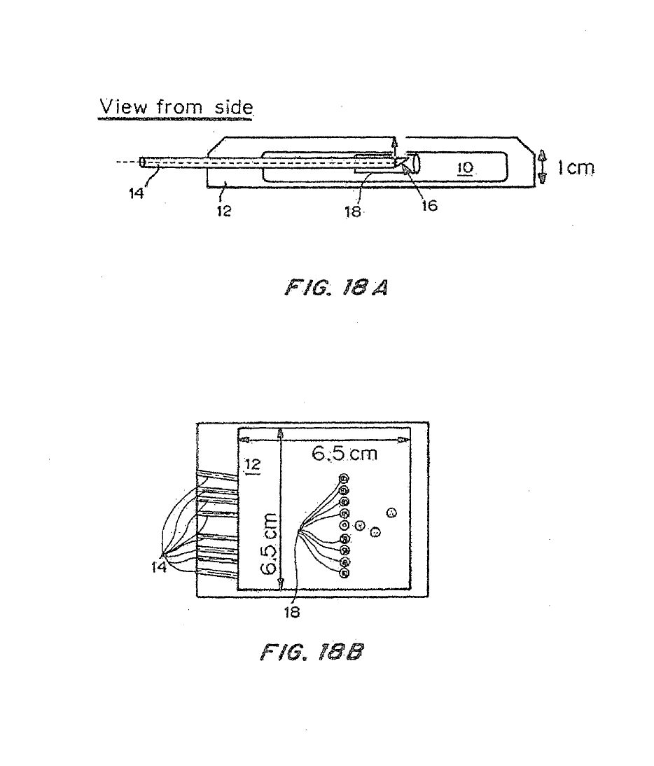

The optical probe enables noninvasive quantification of the hyperemic response of cutaneous and subcutaneous tissue. The design of the probe enables measurement of tissue oxygenation at multiple depths under two conditions: (1) while bearing the weight of the patient (loaded tissue) and (2) when the skin is not bearing weight (unloaded tissue). As shown in FIGS. 18a and 18b, the probe 10 was integrated into a thin silicone pad 12 (approximately 15×15×0.7 cm) that can be gently affixed to patients with medical tape or adhesive and will remain in contact with the sacrococcygeal skin while patients are in both lateral and supine positions.

As shown in FIG. 18a, a probe 10 with optical fibers 14 was inserted into the pad 12. The small prisms 16 were affixed to the end of each optical fiber to turn the light 90 degrees through a hole 18 in the top of the pad 12 which will be in contact with the patient. This arrangement is necessary to minimize the thickness of the pad 12, since the critical bending radius of optical fibers 14 is several centimeters. Prototypes were created by gluing right-angle prisms 16 with 2 mm edges to standard optical fibers 14 that have 2.5 mm metallic ferrules on their ends. Transmission of light through the fibers 14 with glued prisms 16 is consistently 90% of the transmission through the original fibers 14. To protect the ends of these fibers 14, the ferrule/prism complexes were enclosed within 4 mm-diameter metal cylinders 18 with holes to allow light transport to/from the prism. The metal cylinders 18 and a portion of the optical fiber 14 were embedded within the silicone pad 12, as illustrated in FIG. 18b, and the pad 12 provided a smooth, uniform surface for contact with patients. The probe immobilized 10 optical fibers (2 detector and 8 source fibers) that transport light to/from the device components and tissue. The detector fibers were at fixed distances from the source fibers, ranging from 4-20 mm.

This spectroscopy device enables users to objectively classify existing pressure ulcers that present as intact skin with redness or discoloration (i.e., Stage 1 versus deep tissue injury (DTI). The spectroscopy device also allows for the early screening for reversible deep tissue injury by identifying a pre-ulcerative state in high-risk patients. The spectroscopy device allows for assessment of tissue damage at multiple depths up to 1 cm and may be non-invasive. The scans may be performed in as few as 5-10 seconds per scan. In addition, by using the flexible source—detector separation distances of the spectroscopy device described above, multiple locations may be scanned without moving the spectroscopy device. As desired, the contact probe described above may be used to obtain faster measurements and less motion artifacts.

### EXAMPLE 4

### Correlation of Measurements of Surface and Deep Tissue Optical Properties

The observed/applied pressure could be correlated with the measurements of both surface and deep tissue optical properties to develop pressure gauges correlating the measurements with the observed/applied pressures for both contact and non-contact measurement options.

A probe embedded in a silicone mat, shown in FIG. 18b, was used. An elastic abdominal binder (DAL410-Dale Medical Products, Inc.) was wrapped around the entire optical probe including the silicone mat. Optical fibers were fed through a slit that was cut in the abdominal binder. The binder was then fastened around the subjects' waists so that probe remained in close contact with the subjects' sacrococcygeal skin without slipping for the duration of the measurement protocol. The measurement protocol started with baseline measurements on the sacrococcygeal area while the subject was in the lateral position (laying on their sides) for 1-2 minutes. Next, measurements were taken continuously while the subject was moved into supine position with the probe pressed between their sacrococcygeal skin and the bed to induce ischemia for approximately 20 minutes. Lastly, the subject was moved from the supine position back to the lateral position while optical measurements were continued for another 3-5 minutes.

The system was validated using optical phantoms and an in vivo porcine burn model.

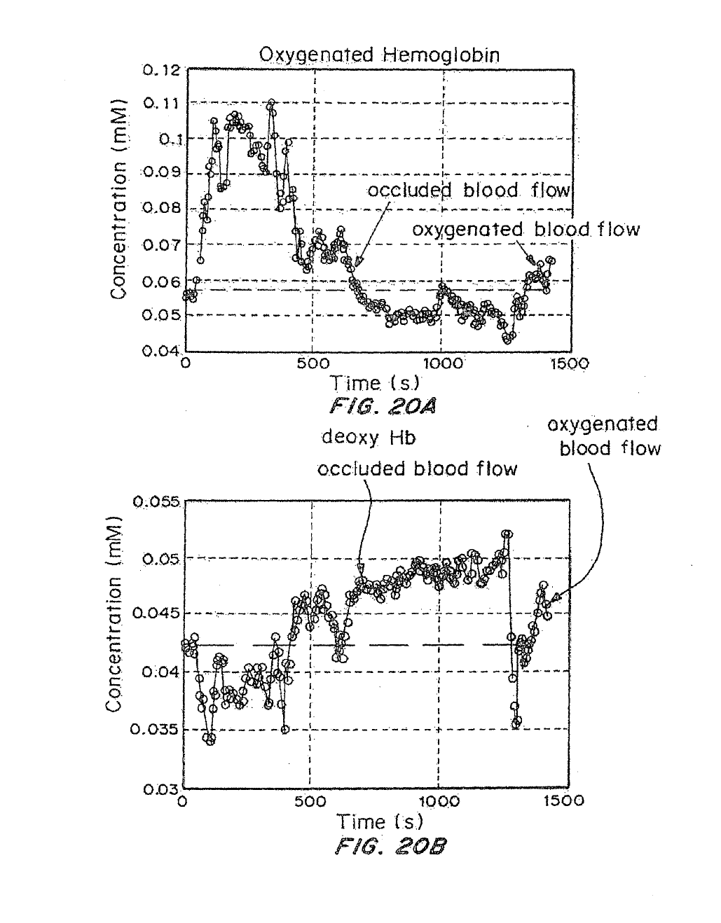

FIGS. 20a and 20b show the calculated concentrations of oxygenated hemoglobin ([HbO]) and deoxygenated hemoglobin ([Hb]) throughout the duration of one representative experiment. As observed, while the subject was in supine position with occluded blood flow, the amount of oxygenated blood decreased as the surrounding tissue metabolized the available oxygen. Simultaneously, the amount of deoxygenated blood increased. Upon moving the patient from supine to lateral position, oxygenated blood rushed into the area, increasing the amount of oxygenated hemoglobin and displacing some of the deoxygenated blood (resulting in a decrease in [HB]).

The healthy tissue could be distinguished from tissue with early pressure ulceration or deep tissue injury based on differences in way oxy- and deoxy-hemoglobin concentrations respond to the protocol of (1) the patient laying in lateral position with unloaded sacrococcygeal skin, followed by (2) ischemia induced by the patient laying in supine position, followed by (3) reperfusion when the patient moves back to lateral position.

Those skilled in the art also will readily appreciate that many additional modifications are possible in the exemplary embodiment without materially departing from the novel teachings and advantages of the invention. For example, those skilled in the art will appreciate that other light source—detector configurations are certainly possible with appropriate adjustments in the optical system. Also, those skilled in the art will appreciate that the spectroscopy device described herein may be used to predict healing of chronic wounds, burns, and surgical flaps, provide objective information to direct initial treatments, assess the wound healing process and effectively evaluate the efficacy of treatments, and assess the extent of sub-surface tissue damage in burn wounds. Accordingly, any such modifications are intended to be included within the scope of this invention as defined by the following exemplary claims.

What is claimed:

1. A device for measuring tissue damage, comprising:
an opto-electronics module including an RF signal source that modulates the intensity of at least one laser diode to provide modulated light at different frequencies, and an optical switch that sequentially switches between respective laser diodes and a no light position;
an optical probe that applies modulated light at a constant modulation frequency at multiple source—detector separations or applying modulated light at multiple modulation frequencies at a single source—detector distance from said opto-electronics module to the tissue of a subject;
at least one detector that detects light scattered by the tissue in response to said modulated light; and
a processing unit responsive to outputs of said at least one detector that calculates optical properties and hemoglobin concentrations within the tissue for correlation with a state of the tissue at different depths beneath the skin.

2. A device as in claim 1, wherein said probe comprises source lenses and detector lenses for applying said modulated light to the tissue in a non-contact manner and detecting scattered light in a non-contact manner, respectively.

3. A device as in claim 1, wherein said probe comprises first optical fibers that apply said modulated light directly to the tissue of the subject and second optical fibers that contact the tissue of the subject to detect the scattered light.

4. A device as in claim 1, wherein said probe comprises source lenses for applying said modulated light to the tissue in a non-contact manner and optical fibers that contact the tissue of the subject to detect the scattered light.

5. A device as in claim 1, wherein said probe comprises optical fibers for applying said modulated light directly to the tissue and detector lenses that detect scattered light in a non-contact manner.

6. A device as in claim 3, wherein said optical probe is inserted into a pad and prisms are affixed to an end of each of said first optical fibers to turn light from said first optical fibers 90 degrees through a hole in a side of said pad arranged to contact the subject.
